(19) United States
(12) Patent Application Publication
(10) Pub. No.: US 2006/0148841 A1
(43) Pub. Date: Jul. 6, 2006

(54) Pharmaceutical composition comprising combination of non-alkaloid and alkaloid-based component for treating skeletal muscle spasm

(76) Inventor: James E. Lundeen, Cleveland Heights, OH (US)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 11/066,611

(22) Filed: Feb. 25, 2005

Related U.S. Application Data
(60) Provisional application No. 60/547,943, filed on Feb. 26, 2004.

(51) Int. Cl. A61K 31/4745

(52) U.S. Cl. 514/291; 514/305

(57) ABSTRACT

A medicinal composition that may include an effective amount of a non-alkaloid and an alkaloid-based skeletal muscle relaxant are provided. A method that includes administering the medicinal composition to a human in an amount effective to treat a muscle spasm is provided. The medicinal composition may include an alkaloid-based and a non-alkaloid-based skeletal muscle relaxant. A kit for treating a muscle spasm, and a system for treating the same are also provided.

### CROSS REFERENCE TO RELATED APPLICATION

The present application claims priority under 35 U.S.C. § 119(e) to U.S. Provisional Patent Application No. 60/547,943, which was filed on Feb. 26, 2004, the disclosure of which is hereby incorporated by reference.

### BACKGROUND

1. Technical Field

The present invention relates to a method for treating a muscle spasm using a medicine including a combination of an alkaloid-based and a non-alkaloid component. The present invention particularly relates to a medicinal composition including a polycyclic β-carboline alkaloid and a non-alkaloid skeletal muscle relaxant for use in the treatment of a muscle spasm.

2. Related Art

A normal voluntary muscle contraction begins when electrical signals are sent from the brain through the spinal cord along nerve cells called motor neurons to a muscle. At the muscle, a chemical is released by the motor neuron to stimulate the internal release of calcium ions from stores within the muscle cell. These calcium ions interact with a muscle protein within the muscle cell to cause proteins (e.g., actin and myosin) to slide past one another. This sliding motion pulls fixed ends of the protein closer together, and thereby shortens the muscle cell or muscle fiber length and, ultimately, the muscle itself. Recapture of calcium, and the unlinking of the actin and myosin proteins, subsequently allows the muscle fiber to relax.

An abnormal contraction may be caused by abnormal activity at any stage in a muscle contraction process. For example, mechanisms within the brain and the rest of the central nervous system regulate contraction, and an interruption of these mechanisms may result in a muscle spasm. Alternatively, overly sensitive motor neurons may fire below their normal thresholds, the muscle membrane itself may be overly sensitive, thus causing contraction without stimulation. Alternatively, calcium ions may not be recaptured quickly enough, resulting in prolonged muscle contraction.

Factors that may contribute to muscle spasms may include dehydration and salt depletion; metabolic disorders that affect the energy supply in muscle, such as, for example, deficiencies of myophosphorylase (McArdle's disease), phosphorylase, β-kinase, phosphofructokinase, phosphoglycerate kinase, and lactate dehydrogenase; myotonia, such as myotonic dystrophy, myotonia congenita, paramyotonia congenita, and neuromyotonia; and/or fasciculations, caused by, for example, fatigue, cold, medications, metabolic disorders, nerve damage, or neurodegenerative disease, including amyotrophic lateral sclerosis.

Quinine has been used as a remedy both for relief of muscle cramps and as an anti-malarial agent. However, the use of quinine as a remedy for treatment of muscle cramps is known to frequently include undesirable side effects, such as ringing in the ears, decreased hearing, vertigo, blurred vision, nausea, vomiting, diarrhea, abdominal pain, flushing or itching of the skin, headache, and/or fever.

Accordingly, a need remains in the medical arts for a medicinal composition useful for treating muscle spasms that takes advantage of the benefits of anti-spasmodics such as quinine but which avoids or reduces some or all of the various side effects associated with such anti-spasmodics.

### SUMMARY

In one embodiment, the present invention relates to a medicinal composition including an effective amount of a polycyclic β-carboline alkaloid, and an effective amount of a non-alkaloid skeletal muscle relaxant.

In another embodiment, the present invention relates to a method including administering to a patient in need thereof a medicinal composition including an effective amount of a polycyclic β-carboline alkaloid, and an effective amount of a non-alkaloid skeletal muscle relaxant.

In another embodiment, the invention further relates to a kit for treating a muscle spasm, and a system for treating the same, in which the kit includes and the system employs a medicinal composition including an effective amount of a polycyclic β-carboline alkaloid, and an effective amount of a non-alkaloid skeletal muscle relaxant.

In accordance with various embodiments of the present invention, a composition and a method are provided which allow use of a polycyclic β-carboline alkaloid, such as quinine, together with a non-alkaloid skeletal muscle relaxant for treating muscle spasms, in which the combination takes advantage of the benefits of anti-spasmodics such as quinine but which avoids or reduces some or all of the various side effects associated with such anti-spasmodics.

### BRIEF DESCRIPTION OF THE DRAWINGS

FIG. 1 is a block diagram showing a method comprising an embodiment in accordance with the invention.

### DETAILED DESCRIPTION

In one embodiment, the present invention relates to a method for treating a muscle spasm. In one embodiment the method includes administering a medicine including a combination of a polycyclic β-carboline alkaloid and a non-alkaloid-based skeletal muscle relaxant to a patient in need thereof, e.g., a patient suffering from muscle spasms. In one embodiment, the present invention relates to a medicine useful for the treatment of a muscle spasm, which includes a polycyclic β-carboline alkaloid and a non-alkaloid-based skeletal muscle relaxant.

As used herein, an effective amount is an amount greater than zero that prevents, reduces or eliminates a muscle spasm, for example, a skeletal muscle spasm, in a human suffering from muscle spasms, without resulting in undue side effects. Representative and exemplary amounts believed to be effect amounts are provided herein.

A spasm refers to any uncontrolled or uncontrollable muscle contraction. Skeletal muscle spasms of particular interest herein include muscle spasms in the upper extremities, torso, upper torso, and pelvic floor, as well as spastic torticollis and other spinal level spasms. In one embodiment, the muscle spasms to be treated with the medicine of the present invention are limited to the torso and upper extremities. In one embodiment, the treatment is limited to cramps and muscle spasms in the upper part of the body, i.e., in the torso, upper torso, such as in the arms, neck, shoulders, intercostal, upper abdomen, diaphragm and/or chest.

In one embodiment, the composition of the invention includes a polycyclic β-carboline alkaloid. In one embodiment, a suitable polycyclic β-carboline alkaloid includes chinchona alkaloids. Suitable chinchona alkaloids include, for example, an isomer selected from (+) isomers: cinchonine, quinidine, dihydrocinchonine, and dihydroquinidine; and (−) isomers: cinchonidine, quinine, dihydrocinchonidine, and dihydroquinine. In one embodiment, the polycyclic β-carboline alkaloid includes quinine.

In one embodiment, the alkaloid may be present or used in the form of a salt, an analog, a metabolite, or a derivative of such alkaloid. In one embodiment, the polycyclic β-carboline alkaloid is quinine salt, such as quinine hydrochloride or quinine dihydrochloride, or quinine sulfate or quinine bisulphate.

The alkaloid quinine may be obtained from the bark of the plant Cinchona officinalis (which is sometimes confusingly also referred to as “quinine”) and related species of Cinchona, and is shown structurally below.

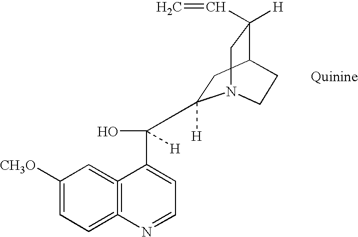

Quinine may be obtained commercially as a generic pharmaceutical from, for example, Cox Arthur H & Co Ltd (North Devon, England). Quinine may also be obtained by the following process. Cinchona bark may be mixed with lime juice (acidic), and then extracted with paraffin oil to form a liquid. The liquid may be filtered, and mixed and shaken in combination with sulfuric acid to form an acidified liquid. The acidified liquid may be mixed with sodium carbonate to neutralize the acidified liquid. A crystallization process may form quinine sulfate as a crystallized product. Neat quinine (i.e., the free base form) may be obtained by organic extraction from an ammoniacal solution of quinine sulfate or other salt. As is known, the crystalline form is more stable and water soluble.

Other alkaloids that may be useful include chloroquine, hydroxychloroquine, and primaquine.

Quinine and polycyclic β-carboline alkaloids have been used for many years in treatment of muscle spasms, malaria and malaria-type diseases. It is known that in the doses that have been used for such treatments, quinine may cause undesirable side effects such as ringing in the ears, headache, dizziness, changes in blood pressure. In one embodiment of the present invention, by combining quinine or other polycyclic β-carboline alkaloid with a non-alkaloid skeletal muscle relaxant, the dosage of quinine used can be reduced, and in one embodiment, a synergistic effect is obtained by the combination.

In one embodiment, a daily dosage of the polycyclic β-carboline alkaloid may in a range of from about 200 milligrams (mg) to about 300 mg, less than about 200 mg, or greater than about 300 mg. In one embodiment, the polycyclic β-carboline alkaloid daily dose may be in a range of from about 0.1 mg to about 1 mg, from about 1 mg to about 10 mg, from about 10 mg to about 25 mg, from about 25 mg to about 50 mg, from about 50 mg to about 75 mg, from about 75 mg to about 100 mg, from about 100 mg to about 125 mg, from about 125 mg to about 150 mg, or from about 150 mg to about 175 mg. In one embodiment, quinine is provided in tablets containing 200 mg or 300 mg of quinine sulfate, and the usual dosage is one tablet taken from one to three times a day. Here and throughout the specification and claims, range limitations may be combined and interchanged.

During use, in one embodiment the polycyclic β-carboline alkaloid may perform one or more of the following functions: increase the tension response to a single, or few, maximal stimuli; increase the refractory period of skeletal muscle such that the responses to the tetanic stimulation may be reduced; decrease the excitability of the motor end-plate such that response to repetitive nerve stimulation and/or to acetylcholine may be reduced. In one embodiment, the polycyclic β-carboline alkaloid may antagonize the action of physostigmine on skeletal muscle. That is, in one embodiment the polycyclic β-carboline alkaloid may simulate curare in function.

In one embodiment, the polycyclic β-carboline alkaloid is used in combination with one or more suitable non-alkaloid-based skeletal muscle relaxant. Because the spasm initiating action may originate in more than one location, and/or may be the result of one or causes, the polycyclic β-carboline alkaloid may be effective to treat the spasm initiating action at one location, or from one causation, while a non-alkaloid-based skeletal muscle relaxant may be effective or helpful to treat the spasm at, or originating from, a second, different location, or from another, different causation.

In one embodiment, the amount of polycyclic β-carboline alkaloid in a daily dosage may be decreased in response to an increase in one or more non-alkaloid-based skeletal muscle relaxants in the daily dosage. Conversely, the non-alkaloid-based skeletal muscle relaxant daily dosage may be decreased in response to an increase in the amount of polycyclic β-carboline alkaloid in the daily dosage.

Quinine and the polycyclic β-carboline alkaloid are not CNS active muscle relaxants, but instead are peripherally acting. Although a small amount of quinine and/or the polycyclic β-carboline alkaloid may cross the blood-brain barrier, by far the most important site of action of these agents is peripheral, not central.

In one embodiment, a suitable non-alkaloid-based skeletal muscle relaxant includes cyclobenzaprine hydrochloride, which may be commercially available from McNeil Corporation (Fort Washington, Pa.) as FLEXERIL®. Cyclobenzaprine hydrochloride may be combined with a polycyclic β-carboline alkaloid such as quinine in a medicine according to embodiments of the invention. In one embodiment, a useful dosage of cyclobenzaprine hydrochloride is 10 milligrams, usually 4 times a day. Other dosages may be appropriate. In one embodiment, a dosage upper limit is about 40 milligrams a day.

In one embodiment, suitable non-alkaloid-based skeletal muscle relaxant includes an enzymatic protein antagonist, such as botulinum toxin; a central nervous system acting skeletal muscle relaxant, such as benzodiazepine; and other non-alkaloid-based skeletal muscle relaxants, and combinations or mixtures of two or more thereof.

In one embodiment, the non-alkaloid skeletal muscle relaxant includes botulinum toxin. Botulinum toxin compound is produced by the bacterium Clostridium botulinum, and enzymatically breaks down one of the fusion proteins that allow neurons to release acetylcholine at a neuromuscular junction. Botulinum toxin is popularly known as BOTOX®, available from Allergan, Inc. The botulinum toxin may prevent, reduce or eliminate the release of acetylcholine from a presynaptic neuron. By interfering with nerve impulses in this way, the botulinum toxin may cause decreased activity or paralysis of muscles. In one embodiment, the botulinum toxin is administered by injection directly into the target muscle. In one embodiment, the treatment ranges from about 150 units to about 400 units, and in one embodiment from about 200 units to about 300 units. One unit of BOTOX® corresponds to the calculated median intraperitoneal lethal dose (LD₅₀) in mice. The injections are applied as needed, periods between injections can range from a few weeks to a few months. In an embodiment comprising botulinum toxin as the non-alkaloidal skeletal muscle relaxant, the botulinum toxin may be separately co-packaged with the polycyclic β-carboline alkaloid component, and may be injected directly into the muscle.

Suitable central nervous system (CNS) acting skeletal muscle relaxants may include a benzodiazepine. A variety of benzodiazepines are known, and any may be suitably selected for use with the present invention. Benzodiazepines also may function as a hypnotic, anxiolytic, anticonvulsive, antiepileptic, amnestic in addition to a muscle relaxant. The CNS acting skeletal muscle relaxants may act on the GABA receptor (GABAA), the activation of which may reduce higher neuronal activity. In one embodiment, the CNS acting skeletal muscle relaxants, such as benzodiazepine, may be used for short-term relief for an acute spasm.

In one embodiment, the CNS acting skeletal muscle relaxant comprises clonazepam. Clonazepam is available in dosages of 0.25 mg and 0.5 mg, and may be taken two or three times a day.

In one embodiment, the CNS acting skeletal muscle relaxant comprises or further comprises alprazolam (“XANAX®”); bromazepam; chlordiazepoxide (“LIBRIUM”); clorazepate; diazepam (“VALIUM®”); flunitrazepam (“ROHYPNOL®”); lorazepam (“ATIVAN®”); medazepam; midazolam (“VERSED®”); oxazepam; prazepam; temazepam; triazolam (“HALCION®”), or a mixture of two or more of the foregoing. In one embodiment, one or more of the foregoing may be combined with one or more of the other non-alkaloid skeletal muscle relaxants disclosed herein. Commercially available brand name trademarks are shown in parentheses.

Alprazolam is available in 0.25 mg and 0.5 mg tablets, and is normally taken two or three times a day.

Bromazepam is available in 3 mg, 6 mg and 10 mg tablets, and may be taken two to four times a day.

Chlordiazepoxide is available in 5 mg, 10 mg and 25 mg tablets, and may be taken three or four times a day.

Clorazepate is available in 7.5 mg and 15 mg tablets, and may be taken two to four times a day.

Diazepam is available in 2 mg, 5 mg and 10 mg tablets, and may be taken two to four times a day.

Flunitrazepam is available in 0.5 mg and 2 mg tablets, and may be taken two or three times a day.

Lorazepam is available in 1 mg, 2 mg and 3 mg tablets, and may be taken two or three times a day.

Medazepam is available in 5 mg and 10 mg tablets, and may be taken two or three times a day.

Midazolam is usually given by injection or IV; usual dosages are from 2.5 to 5 mg per dose, given, for example, by slow push IV injection over 2-3 minutes.

Oxazepam is available in 10 mg and 20 mg tablets, and may be taken three or four times a day.

Prazepam is available in 10 mg, 20 mg and 30 mg tablets, and may be taken two or three times a day.

Triazolam is available in 0.125 mg and 0.25 mg tablets, and may be taken at bedtime as a sleep aid.

Temazepam is available in 7.5 mg and 15 mg tablets, and may be taken at bedtime as a sleep aid.

Other suitable CNS skeletal muscle relaxants may include one or more of carisoprodol, chlorphenesin, chlorzoxazone, metaxalone, methocarbamol, orphenadrine, and analogs, derivative, metabolites and salts thereof. Brand name versions may be obtained commercially, for example, orphenadrine citrate as NORFLEX®from 3M Pharmaceuticals Inc. (St. Paul, Minn.). NORFLEX is supplied in 100 mg tablets, may be taken twice a day. In one embodiment, the CNS acting skeletal muscle relaxant daily dose may be in a range of from about 100 mg to about 200 mg.

In one embodiment, other known muscle relaxers may be used, including: SOMA®, Skelaxin and Zanaflex.

Suitable dosages of and dosage regimens for the foregoing skeletal muscle relaxants can be determined by those of ordinary skill in the art based on known and usual dosages for similar or related usages.

The foregoing muscle relaxers may be used in the compositions of the present invention in appropriate dosages, based on the usual dosages of these drugs, and based on the intended use herein. As noted, in at least some of the embodiments of the present invention, a synergistic effect is obtained by use of the disclosed combination of a polycyclic β-carboline alkaloid and a non-alkaloid skeletal muscle relaxant. In such embodiment, the usual dosage of either or both of the disclosed combination of a polycyclic β-carboline alkaloid, and a non-alkaloid skeletal muscle relaxant, particularly of the non-alkaloid skeletal muscle relaxant, may be reduced from the usual dosage of either used alone.

In one embodiment, the medicinal composition consists essentially of an effective amount of a polycyclic β-carboline alkaloid and an effective amount of a non-alkaloid skeletal muscle relaxant. In one embodiment, the medicinal composition consists essentially of an effective amount of a chinchona alkaloid, and an effective amount of a non-alkaloid skeletal muscle relaxant. In one embodiment, the medicinal composition consists essentially of an effective amount of a chinchonine, quinidine, dihydrocinchonine, or dihydroquinidine, or an analog, metabolite, or derivative thereof or a mixture of any two or more thereof, and an effective amount of a non-alkaloid skeletal muscle relaxant. In one embodiment, the medicinal composition consists essentially of an effective amount of cinchonidine, quinine, dihydrocinchonidine, or dihydroquinine, or an analog, metabolite, or derivative thereof or a mixture of any two or more thereof, and an effective amount of a non-alkaloid skeletal muscle relaxant. In one embodiment, the medicinal composition consists essentially of an effective amount quinine, or an analog, metabolite, or derivative thereof, and an effective amount of a non-alkaloid skeletal muscle relaxant. In one embodiment, the medicinal composition consists essentially of an effective amount quinine or quinine salt, and an effective amount of a non-alkaloid skeletal muscle relaxant.

In one embodiment, the combination of the polycyclic β-carboline alkaloid and the non-alkaloidal skeletal muscle relaxant result in a synergistic muscle relaxation, in which the total muscle relaxation effect is significantly greater than the action of either one alone or of the additive effect that would be expected from the combination of the two ingredients.

In one embodiment, the polycyclic β-carboline alkaloid is quinine. In one embodiment, the polycyclic β-carboline alkaloid consists essentially of quinine or a derivative thereof. In one embodiment, the medicinal composition consists essentially of an effective amount of a polycyclic β-carboline alkaloid, and an effective amount of a non-alkaloidal skeletal muscle relaxant. In one embodiment, the medicinal composition consists essentially of an effective amount of a polycyclic β-carboline alkaloid, and an effective amount of clonazepam. In one embodiment, the medicinal composition consists essentially of an effective amount of a polycyclic β-carboline alkaloid, and an effective amount of botulinum toxin. In one embodiment, the medicinal composition consists essentially of an effective amount of a polycyclic β-carboline alkaloid, and an effective amount of a benzodiazepine. In one embodiment, the medicinal composition consists essentially of an effective amount of a polycyclic β-carboline alkaloid, and an effective amount of cyclobenzaprine hydrochloride. In one embodiment, the medicinal composition consists essentially of an effective amount of a polycyclic β-carboline alkaloid, and an effective amount of alprazolam, bromazepam, chlordiazepoxide, clorazepate, diazepam, flunitrazepam, lorazepam, medazepam, midazolam, oxazepam, prazepam, temazepam, or triazolam, or an analog, derivative, metabolite, or salt thereof, or a mixture of any two or more thereof. In one embodiment, the medicinal composition consists essentially of an effective amount of a polycyclic β-carboline alkaloid, and an effective amount of carisoprodol, chlorphenesin, chlorzoxazone, metaxalone, methocarbamol, or orphenadrine, or an analog, derivative, metabolite or salt thereof or a mixture of any two or more thereof.

In one embodiment according to the present invention, the alkaloid-based medicine may include one or more additional materials, in combination with the polycyclic β-carboline alkaloid and the non-alkaloidal skeletal muscle relaxant. Suitable additional material may include one or more of an analgesic; an anti-inflammatory; a hypnotic; a narcotic; an opioid, or opioid agonist; an anti-depressant; a proton pump inhibitor; a beta blocker; a barbiturate; a laxative; an anti-epileptic; and a hormone.

In one embodiment, the composition further comprises an analgesic. In one embodiment, the analgesic is a non-steroidal anti-inflammatory (NSAID), and in another embodiment, the analgesic is a narcotic. Suitable narcotics may include opioid agonists, such as PERCOCET® (oxycondone plus acetaminophen), which is commercially available from Endo Laboratories, Inc. (Chadds Ford, Pa.). PERCOCET®, for example, is provided in dosage sizes of 2.5 mg, 5 mg, 7.5 mg and 10 mg with, for example, the 5 mg dosage being most often prescribed, and is normally taken once every six hours, i.e., four times a day.

Suitable proton pump inhibitors may include omeprazole or 5-methoxy-2-[[(4-methoxy-3,5-dimethyl-2-pyridinyl) methyl]sulfinyl]-1H-benzimidazole, which may be commercially available from AstraZeneca LP (Wilmington, Del.) as PRILOSEC®, and lansoprazole, which is commercially available from TAP Pharmaceutical Products Inc. (Lake Forrest, Ill.) as PREVACID®. In one embodiment, the proton pump inhibitor is PRILOSEC®, and the daily dose may be a single dose, or two or more doses, in a range of from about 20 mg to about 200 mg, or from about 40 mg to about 150 mg total daily dose. In one embodiment, the proton pump inhibitor is PREVACID®, and the daily dose may be from 1 to 3 times a day in a range of from about 15 mg to about 60 mg, or from about 30 mg to about 45 mg total daily dose.

Suitable NSAIDs include virtually any known NSAID. NSAIDs comprise a large class of drugs with many different options. Ibuprofen and naproxen are exemplary NSAIDs. The recommended dose of ibuprofen is 400 mg every eight hours, although prescription doses can be as high as 800 mg every eight hours. The recommended dose of naproxen ranges from 75 mg every eight hours to a maximum daily dose of 1000 mg, usually taken in two doses of 500 mg each. Another group of NSAIDs are the COX-2 inhibitors, such as BEXTRA®, CELEBREX® and VIOXX®. BEXTRA® is available in both 10 mg and 20 mg tablets, taken once or twice a day. CELEBREX® is available in 100 mg, 200 mg and 400 mg tablets, taken once or twice a day. VIOXX® is available in 12.5 mg, 25 mg and 50 mg tablets, taken once a day. (At the time of filing this application VIOXX® has been voluntarily withdrawn from the market. It is not known whether it will be made available in the future.) In an embodiment including a NSAID (as with the other additional ingredients), the quantity of NSAID per dose should be adjusted to correspond to the dosage regimen selected for the primary ingredients (the polycyclic β-carboline alkaloid and the non-alkaloid skeletal muscle relaxant), in order to provide an effective but safe amount of the NSAID.

In one embodiment, the medicine further includes a beta-blocker, such as atenolol, which is commercially available from Medley Pharmaceuticals, Ltd (Maharashtra, India) as TENORMIN®. Atenolol is a synthetic, beta1-selective (cardioselective) adrenoreceptor blocking agent or “beta-blocker”, that may be chemically described as benzeneacetamide, 4-[2′-hydroxy-3′-[(1-methylethyl) amino] propoxy] benzeneacetamide. Atenolol may block the action of the sympathetic nervous system. Because the sympathetic nervous system controls or influences the pace of the heart beat, blocking the action of these nerves can reduce the heart rate. Atenolol may reduce the force and rate of heart muscle contraction, lower blood pressure. Where tachycardia may be caused, a beta-blocker such as atenolol may be used to maintain the heart rate in a desired range. In one embodiment, the beta blocker daily dose may be in a range of from about 50 mg to about 200 mg, or from about 50 mg to about 100 mg per day, given in a single dose.

In one embodiment, suitable additional material may include one or more of carbazamine (TEGRETOL® from Novartis, Inc.), ethosuximinde (ZARATONIN™ from Pfizer, Inc. (New York, N.Y.), felbamate (FELBATOL® from Wallace Laboratories (Cranbury, N.J.)), gabapentin (NEURONTIN® from Pfizer, Inc.), lamotrigine (LAMICTAL® from GlaxoSmithKline, Inc. (London, England)), levetiracetam (KEPPRA® from UCB Pharma, Inc. (Smyrna, Ga.)), phenobarbital (Generically available), Phenytoin (DILANTIN® from Pfizer, Inc.), Tiagabine (GABITRIL® from Cephalon, Inc. (Guildford, England)) topiramate (TOPAMAX® from Ortho-McNeil Pharmaceuticals, Inc. (Raritan, N.J.), valproate (DEPAKOTE® from Abbott Laboratories, Inc. (Abbott Park, Ill.), or zonisamine (ZONEGRAN® from Eisai, Inc. (Tokyo, Japan)). In one embodiment, suitable additional materials may include one or both of staurosporine or tetrodotoxin.

In one embodiment, suitable additional material may include one or more tricyclic antidepressants. In one embodiment, the tricyclic antidepressant may function at the cellular level to inhibit re-uptake of the exocytosed monoamines serotonin and noradrenaline. In one embodiment, the tricyclic antidepressant may stimulate or enhance the production of inositol 1,4,5-trisphosphate. In one embodiment, the tricyclic antidepressant may stimulate or enhance phospholipase C activity. Phospholipase C enhancement or stimulation may affect production of a protein kinase C activator, such as diacylglycerol, which may affect the synaptic strength of neurons in a protein kinase C-dependent manner. In one embodiment, the tricyclic antidepressant may stimulate or enhance the spontaneous vesicular release of glutamate. In one embodiment, the tricyclic antidepressant may perform two or more of the above functions. The tricyclic antidepressant may function to affect one or more of serotoninergic cells, noradrenergic cells, and/or modify the activity of glutamatergic neurons.

In one embodiment, the medicinal composition of the present invention is provided in the form of a tablet, gelcap, or other suitable orally administrable form. In one embodiment, each dosage of the medicinal composition contains a quantity of the polycyclic β-carboline alkaloid and the non-alkaloid skeletal muscle relaxant effective when administered in intervals of 4 hours, 8 hours, 12 hours or 24 hours.

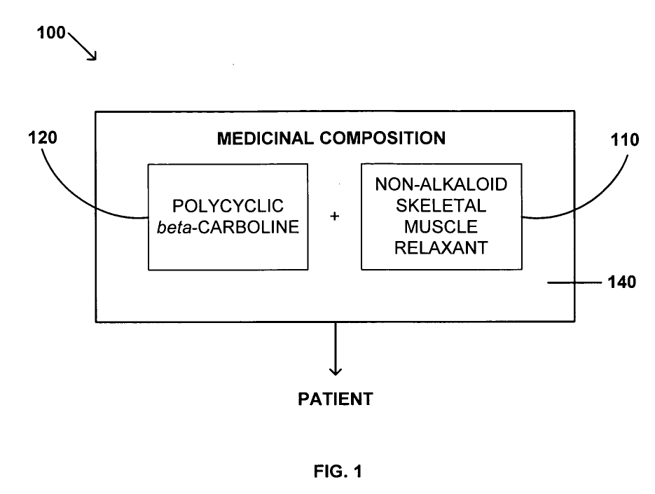

FIG. 1 schematically illustrates an embodiment of a method in accordance with the present invention. As shown in FIG. 1, the method 100 includes combining a non-alkaloid skeletal muscle relaxant 110 with a polycyclic β-carboline alkaloid 120 to form a medicinal composition 140. An effective amount of the medicinal composition 140 may be administered to a patient that suffers from skeletal muscle spasms. As shown, the non-alkaloid skeletal muscle relaxant 110 and the polycyclic β-carboline alkaloid 120 may be combined and administered together to the patient, or in an alternative embodiment (not shown), the components 120, 140 of the medicinal composition may be administered individually to the patient 150.

Throughout the foregoing specification, dosage forms, amounts and regimens of the various active ingredients have been described primarily in terms of presently existing products in which these active ingredients are included. It is to be understood that when these various active ingredients are employed in the present invention, that the dosage forms, amounts and regimens, including amounts administered in any individual dosage, may be changed and adjusted as needed to correspond to the uses described herein. Persons of ordinary skill in the art can determine appropriate dosage forms, amounts and regimens based on the foregoing specification and the knowledge of such persons.

It is noted that, throughout the specification and claims, the numerical limits of the disclosed ranges and ratios may be combined, and are deemed to include all intervening values. Furthermore, all numerical values are deemed to be preceded by the modifier “about”, whether or not this term is specifically stated.

While the principles of the invention have been explained in relation to certain particular embodiments, and are provided for purposes of illustration, it is to be understood that various modifications thereof will become apparent to those skilled in the art upon reading the specification. Therefore, it is to be understood that the invention disclosed herein is intended to cover such modifications as fall within the scope of the appended claims. The scope of the invention is limited only by the scope of the claims.

What is claimed is:

1. A medicinal composition comprising:
an effective amount of a polycyclic β-carboline alkaloid; and
an effective amount of a non-alkaloid skeletal muscle relaxant.

2. The composition as defined in claim 1 wherein the polycyclic β-carboline alkaloid comprises a chinchona alkaloid.

3. The composition as defined in claim 2 wherein the chinchona alkaloid comprises chinchonine, quinidine, dihydrocinchonine, dihydroquinidine, cinchonidine, quinine, dihydrocinchonidine, or dihydroquinine or an analog, metabolite, or derivative thereof, or a mixture of any two or more thereof.

4. The composition as defined in claim 1 wherein the polycyclic β-carboline alkaloid comprises quinine, an analog, a metabolite, a salt, or a derivative of quinine, or a mixture of any two or more thereof.

5. The composition as defined in claim 1 wherein the polycyclic β-carboline alkaloid is present in a single dosage form in an amount in a range of less than about 200 milligrams, or in a range of from about 200 milligrams to about 300 milligrams, or in a range of from about 300 milligrams to about 500 mg.

6. The composition as defined in claim 1 wherein the polycyclic β-carboline alkaloid is present in a single dosage form in an amount in a range of from about 0.1 milligrams to about 100 milligrams.

7. The composition as defined in claim 1 wherein the non-alkaloid-based skeletal muscle relaxant comprises an enzymatic protein antagonist skeletal muscle relaxant, a central nervous system acting skeletal muscle relaxant or a mixture of any two or more thereof.

8. The composition as defined in claim 7 wherein the enzymatic protein antagonist skeletal muscle relaxant comprises botulinum toxin.

9. The composition as defined in claim 7 wherein the central nervous system acting skeletal muscle relaxant comprises benzodiazepine.

10. The composition as defined in claim 1 wherein the non-alkaloid-based material comprises cyclobenzaprine hydrochloride.

11. The composition as defined in claim 1 wherein the non-alkaloid-based material comprises clonazepam.

12. The composition as defined in claim 1 further comprising one or more of an analgesic, an anti-inflammatory, a hypnotic, a narcotic, an opioid or opioid agonist, an anti-depressant, a proton pump inhibitor, a beta blocker, a barbiturate, a laxative, or a hormone.

13. The composition as defined in claim 1 wherein the non-alkaloid skeletal muscle relaxant comprises alprazolam, bromazepam, chlordiazepoxide, clorazepate, diazepam, flunitrazepam, lorazepam, medazepam, midazolam, oxazepam, prazepam, temazepam, triazolam, carisoprodol, chlorphenesin, chlorzoxazone, metaxalone, methocarbamol, or orphenadrine, or an analog, derivative, metabolite, or salt thereof, or a mixture of any two or more thereof.

14. A method of treating a muscle spasm comprising administering an effective amount of the medicinal composition defined in claim 1 to a patient suffering from muscle spasm.

15. A method of treating a muscle spasm, comprising:
administering a medicinal composition to a human in an amount effective to treat a muscle spasm, the medicinal composition comprising:
a polycyclic β-carboline alkaloid, and
a non-alkaloid skeletal muscle relaxant.

16. The method as defined in claim 15 wherein the polycyclic β-carboline alkaloid comprises a chinchona alkaloid.

17. The method as defined in claim 15 wherein the chinchona alkaloid comprises cinchonine, quinidine, dihydrocinchonine, dihydroquinidine, cinchonidine, quinine, dihydrocinchonidine, or dihydroquinine, or an analog, metabolite, or derivative thereof, or a mixture of any two or more thereof.

18. The method as defined in claim 15 wherein the polycyclic β-carboline alkaloid comprises quinine, an analog, a metabolite, a salt, or a derivative of quinine, or a mixture of any two or more thereof.

19. The method as defined in claim 15 wherein the polycyclic β-carboline alkaloid is present in a single dosage form in an amount in a range of less than about 200 milligrams, or in a range of from about 200 milligrams to about 300 milligrams, or in a range from about 300 milligrams to about 500 milligrams.

20. The method as defined in claim 15 wherein the polycyclic β-carboline alkaloid is present in a single dosage form in an amount in a range of from about 0.1 milligrams to about 100 milligrams.

21. The method as defined in claim 15 wherein the non-alkaloid-based skeletal muscle relaxant comprises an enzymatic protein antagonist skeletal muscle relaxant, or a central nervous system acting skeletal muscle relaxant, or a mixture of any two or more thereof.

22. The method as defined in claim 21 wherein the enzymatic protein antagonist skeletal muscle relaxant comprises botulinum toxin.

23. The method as defined in claim 21 wherein the central nervous system acting skeletal muscle relaxant comprises a benzodiazepine.

24. The method as defined in claim 15 wherein the non-alkaloid-based material comprises cyclobenzaprine hydrochloride.

25. The method as defined in claim 15 wherein the non-alkaloid-based material comprises clonazepam.

26. The method as defined in claim 15 further comprising one or more of an analgesic, an anti-inflammatory, a hypnotic, a narcotic, an opioid or opioid agonist, an anti-depressant, a proton pump inhibitor, a beta blocker, a barbiturate, a laxative, or a hormone.

27. The method as defined in claim 15 wherein the non-alkaloid skeletal muscle relaxant comprises alprazolam, bromazepam, chlordiazepoxide, clorazepate, diazepam, flunitrazepam, lorazepam, medazepam, midazolam, oxazepam, prazepam, temazepam, triazolam, carisoprodol, chlorphenesin, chlorzoxazone, metaxalone, methocarbamol, or orphenadrine, or an analog, derivative, metabolite, or salt thereof, or a mixture of any two or more thereof.

29. A kit comprising:
a medicinal composition effective to treat skeletal muscle spasms, the medicinal composition comprising:
an effective amount of a polycyclic β-carboline alkaloid, and
an effective amount of a non-alkaloid skeletal muscle relaxant.

30. The kit as defined in claim 29 further comprising an instruction set that comprises a dosage regimen for the treatment of muscle spasms.
